SEQUENCE LISTING (1 sequence)

SEQ ID NO: 1 - protein, 493 aa, Homo sapiens
MAAATGPSFWLGNETLKVPLALFALNRQRLCERLRKNPAVQAGSIVVLQGGEETQRYCTDTGVLFLQESFFHWAFGVTEPGCYGVIDVDTGKSTLFVPRLPASHATWMGKIHSKEHFKEKYAVDDVQYVDEIASVLTSQKPSVLLTLRGVNTDSGSVCREASFDGISKFEVNNTILHPEIVESRVFKTDMELEVLRYTNKISSEAHREVMKAVKVGMKEYGLESLFEHYCYSRGGMRHSSYTCICGSGENSAVLHYGHAGAPNDRTIQNGDMCLFDMGGEYYSVASDITCSFPRNGKFTADQKAVYEAVLLSSRAVMGAMKPGDWWPDIDRLADRIHLEELAHMGILSGSVDAMVQAHLGAVFMPHGLGHFLGIDVHDVGGYPEGVERIDEPGLRSLRTARHLQPGMVLTVEPGIYFIDHLLDEALADPARASFLNREVLQRFRGFGGVRIEEDVVVIDSGIELLTCVPRTVEEIEACMAGCDKAFTPFSGPK

(19) United States
(12) Patent Application Publication
(10) Pub. No.: US 2026/0055208 A1
(43) Pub. Date: Feb. 26, 2026

(54) COMBINATION OF GARADACIMAB AND PROLIDASE FOR CANCER THERAPY

(71) Applicant: Health Research, Inc., Buffalo, NY (US)

(72) Inventors: Arup BHATTACHARYA, Buffalo, NY (US); Lu YANG, North Chesterfield, VA (US); Yuesheng ZHANG, Henrico, VA (US)

(21) Appl. No.: 19/102,539

(22) PCT Filed: Aug. 14, 2023
(86) PCT No.: PCT/US2023/072144

§ 371 Date: Feb. 10, 2025

Related U.S. Application Data
(60) Provisional application No. 63/371,479, filed on Aug. 15, 2022.

(51) Int. Cl. C07K 16/36; A61K 38/48; A61K 39/00; A61P 35/00

(52) U.S. Cl. CPC C07K 16/36; A61K 38/4813; A61P 35/00; A61K 2039/505; A61K 2039/545; C12Y 304/13009

(57) ABSTRACT

A method for treating cancer is provided. The method includes administering a combination of a Prolidase, also known as peptidase D (PEPD), in combination with a monoclonal antibody or antigen binding fragment thereof that binds with specificity to a clotting factor, such as Factor XII (FXII). A single dose of the monoclonal antibody is sufficient to enable a therapeutic effect of the PEPD.

### CROSS REFERENCE TO RELATED APPLICATIONS

This application claims priority to U.S. provisional application No. 63/371,479, filed Aug. 15, 2022, the entire disclosure of which is incorporated herein by reference.

### FIELD OF THE DISCLOSURE

The present invention relates generally to the field of human health, and in particular to methods for the prophylaxis and treatment of cancers.

### SEQUENCE LISTING

The instant application contains a Sequence Listing which has been submitted in .xml format and is hereby incorporated by reference in its entirety. Said .xml copy was created on Aug. 11, 2023, is named “Y_Zhang_2023_PEPD.xml,” and is 2,446 bytes in size.

### BACKGROUND OF THE INVENTION

There is an ongoing and unmet need for improved compositions and methods for treating cancer. The present disclosure is pertinent to these needs.

### SUMMARY OF THE DISCLOSURE

The present disclosure provides compositions and methods useful for prophylaxis and/or therapy of cancers, including but not necessarily limited to ErbB1-positive and ErbB2-positive cancers.

Prolidase, also known as peptidase D (PEPD), Xaa-Pro dipeptidase or proline dipeptidase, or imidodipeptidase, is a dipeptidase which hydrolyzes dipeptides with proline or hydroxyproline at the carboxy terminus. PEPD is a ubiquitously expressed cytosolic protein and exists as a homodimer (monomeric molecular weight of human PEPD: 54 kD; 493 amino acids as shown in SEQ ID NO:1, which provides the amino acid sequence of human prolidase). The disclosure includes use of PEPD and certain enzymatically inactive variants thereof as described herein in combination with a monoclonal antibody or antigen binding fragment thereof that binds with specificity to a clotting factor, such as Factor XII (FXII).

### BRIEF DESCRIPTION OF THE FIGURES

FIG. 1. Schematic showing that a representative coagulation inhibitor, Enoxaparin (EP), does not inhibit FXII or FXIIa.

FIG. 2. Data showing plasma concentration of a mutated form of PEPD after enoxaparin (EP) administered ip daily (left panel) and the mutated PEPD plasma concentration after a single dose of garadacimab (right panel).

FIG. 3. Data demonstrating that a single dose of garadacimab is as effective as daily dose of enoxaparin in enabling the antitumor activity of PEPD-G278D in an orthotopic HER2-positive breast cancer patient-derived xenograft (PDX) model. Fragments of fresh human HER2-positive PDX (911LJ) tumors from a donor mouse were transplanted orthotopically to the mammary fat pads of 7-8 weeks of female NSG mice. The drink water contained 1 μM estradiol (to support tumor growth). Tumor volumes were measured using a caliper and calculated by length×width²/2. A, a single dose of garadacimab (40 mg/kg ip) was administered on day 13. PEPD-G278D (5 mg/kg ip) three times weekly was started 3 h after garadacimab and the final dose was on day 32. Each value is mean+/−SEM, n=12. B, daily enoxaparin (0.5 mg/kg ip) was started on day 10. PEPD-G278D (5 mg/kg, ip) three times weekly was started on day 12. The final doses of enoxaparin and PEPD-G278D were on day 26. Each value is mean+/−SEM, n=12-14.

FIG. 4. Data demonstrating that a single dose of garadacimab is as effective as daily doses of enoxaparin in enabling the antitumor activity of PEPD-G278D in orthotopic HER2-positive breast HCC1954 tumors. Human HER2 positive breast cancer HCC1954 cells were inoculated to the mammary fat pads of female athymic nude mice (7-8 weeks). Tumor volumes were measured using a caliper and calculated by length×width2/2. A, a single dose of garadacimab (40 mg/kg ip) was administered on day 15. PEPD-G278D (5 mg/kg ip) three times weekly was started on day 16. Final treatment was on day 33. Each value is mean+/−SEM, n=11-12. B, daily enoxaparin (0.5 mg/kg ip) was started on day 11. PEPD-G278D (5 mg/kg ip) three times weekly was started on day 14. Last doses of the agents were on day 30. Each value is mean+/−SEM, n=14.

### DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step, may be made without departing from the scope of the disclosure.

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

This disclosure includes every amino acid sequence described herein and all nucleotide sequences encoding the amino acid sequences. Polynucleotide and amino acid sequences having from 50-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences, and may include other components, as further described below.

The disclosure includes all polynucleotide and all amino acid sequences that are identified herein by way of a database entry. Such sequences are incorporated herein as they exist in the database on the effective filing date of this application or patent.

Data presented herein unexpectedly demonstrates that a single dose of a monoclonal antibody that specifically binds to Factor XII (FXII) extends the time PEPD remains in circulation and enables the PEPD to promote a therapeutic anti-cancer response. Data presented herein also demonstrate that a single dose of a monoclonal antibody that specifically binds to FXII that is administered prior to administration of PEPD inhibits tumor growth in a clinically relevant animal model. The data show that a single dose of the monoclonal antibody is as effective as a daily dose of enoxaparin, and that enoxaparin, does not inhibit FXII or FXIIa.

In one embodiment a described approach to treating cancer is illustrated using a form of PEPD shown in SEQ ID NO:1, where the G at position 278 is shaded, bolded and italicized and represents the location of a G278 mutation which renders the PEPD enzymatically inactive. In embodiments, the mutation is a change of glycine at position 278 to aspartic acid, but other amino acids may also be substituted for the glycine at position 278 of SEQ ID NO:1.

In more detail, it will be apparent from the instant disclosure and the Examples presented herein that the presence of PEPD in circulation can be extended by administering a monoclonal antibody that binds with specificity to FXII, and that a single dose of the monoclonal antibody is therapeutically effective when followed by PEPD administration. In this regard, the anti-cancer effects of PEPD are established in the art, such as is described in PCT publication WO 2015/031119, and PCT publication WO 2016/028956, the disclosures of each of which are incorporated herein by reference. The present disclosure differs from prior approaches in the demonstration that combining PEPD with a signal dose of a monoclonal antibody that binds with specificity to FXII improves the anti-cancer effects of PEPD, and therefore frequent administrations of an alternative anti-coagulant such as enoxaparin is not required. Notably, neither garadacimab nor EP is known to have antitumor activity by itself. In an embodiment, that monoclonal antibody that binds with specificity to FXII is the monoclonal antibody known in the art as Garadacimab.

Any PEPD is expected to be suitable for use in the compositions and methods of the present disclosure. In embodiments, the PEPD is a PEPD produced by a prokaryote or a eukaryote. In embodiments, the PEPD is prokaryotic in origin. In non-limiting embodiments, the PEPD is produced by Pseudoalteromonas haloplanktis (i.e., a PEPD comprising the amino acid sequence under GenBank no. AAA99824.1), or Pyrococcus furiosus (i.e., a PEPD comprising the amino acid sequence under GenBank no. WP_011011876.1). A number eukaryotic PEPD amino acid sequences are also known in the art, including a number of mammalian PEPD amino acid sequences. In embodiments, the PEPD has the sequence of a rodent PEPD, i.e., a mouse or rat, or a non-human primate PEPD, such as chimpanzee or a Rhesus macaque. The amino acid sequence of mouse prolidase is provided under GenBank accession no. NP_032846.2; rat prolidase is provided under NP_001009641.1; Rhesus macaque prolidase is provided under AFJ71215.1; chimpanzee prolidase is provided under NP_001267459.1. The amino acid sequence of human prolidase (PEPD) in SEQ ID NO: 1 is known in the art. SEQ ID NO: 1 and the cDNA sequence encoding it is accessible via GenBank accession no. J04605.1; the amino acid sequence is also provided under GenBank accession number AAA60064. In one illustrative but not limiting embodiment, enzymatically active human PEPD has the sequence of SEQ ID NO:1:

(SEQ ID NO: 1)
MAAATGPSFWLGNETLKVPLALFALNRQRLCERLRKNPAVQAGSIVVLQG
GEETQRYCTDTGVLFLQESFFHWAFGVTEPGCYGVIDVDTGKSTLFVPRL
PASHATWMGKIHSKEHFKEKYAVDDVQYVDEIASVLTSQKPSVLLTLRGV
NTDSGSVCREASFDGISKFEVNNTILHPEIVESRVFKTDMELEVLRYTNK
ISSEAHREVMKAVKVGMKEYGLESLFEHYCYSRGGMRHSSYTCICGSGEN
SAVLHYGHAGAPNDRTIQNGDMCLFDMGGEYYSVASDITCSFPRNGKFTA
DQKAVYEAVLLSSRAVMGAMKPGDWWPDIDRLADRIHLEELAHMGILSGS
VDAMVQAHLGAVFMPHGLGHFLGIDVHDVGGYPEGVERIDEPGLRSLRTA
RHLQPGMVLTVEPGIYFIDHLLDEALADPARASFLNREVLQRFRGFGGVR
IEEDVVVIDSGIELLTCVPRTVEEIEACMAGCDKAFTPFSGPK

In SEQ ID NO: 1, the G at position 278 bolded and italicized and represents the location of a G278D mutation which renders the PEPD enzymatically inactive. In embodiments, the mutation is a change of glycine at position 278 to an amino acid other than aspartic acid.

All of the amino acid and polynucleotide sequences provided under the GenBank accession numbers referenced in this disclosure are incorporated herein by reference as those sequences were available through GenBank on the date of filing of this application. This disclosure also includes all polynucleotides encoding PEPD and all variants of it that are described herein or which would otherwise be known to the skilled artisan given the benefit of the present disclosure.

Rodent (mouse and rat) PEPD amino acid sequences are more than 86% similar to the human sequence, while non-human primate PEPD amino acid sequences, such as the Rhesus macaque, is over 95% similar to the human PEPD amino acid sequence. In embodiments, the PEPD comprises or consists of a human PEPD amino acid sequence. In embodiments, the PEPD used in the compositions and/or methods of the present disclosure is at between at least 85.0% and 99.9%, inclusive, and including all numerals to the first decimal place there between, similar to the sequence of SEQ ID NO:1. In an embodiment, the PEPD comprises an amino acid sequence that is at least 95% similar to the sequence of SEQ ID NO:1.

In various embodiments, the present disclosure includes compositions comprising wild type PEPD (e.g., PEPD of SEQ ID NO:1), or modified PEPD, or a combination thereof. In general, modifications to PEPD suitable for use with the present invention can be determined by those skilled in the art using ordinary techniques, given the benefit of the present description. In embodiments, modified PEPD comprises modifications of SEQ ID NO: 1. The disclosure includes all modifications of SEQ ID NO: 1 so long as the PEPD retains the capability to bind to and cause depletion of ErbB2 from the cell surface. In embodiments, modified PEPD retains the capability to form a homodimer. In embodiments, contacting an ErbB1 or ErbB2-positive cell with a modified (or wild type) PEPD of this disclosure is followed by receptor binding and endocytosis, resulting in receptor depletion. Modified PEPD that maintain some or all of these functional attributes may comprise amino acid insertions, deletions and substitutions.

Wild type PEPD is enzymatically active. Modified PEPD is a PEPD that comprises a change in SEQ ID NO: 1 and can be enzymatically active or enzymatically inactive. In embodiments, enzymatically inactive PEPD can have at least between 0.0%-99.9%, inclusive, and including all digits there between to the first decimal point, less dipeptide hydrolysis activity as compared to a reference PEPD. In one embodiment, an enzymatically inactive PEPD has no more than 0.6% dipeptide hydrolysis activity of a reference PEPD. In one embodiment, the reference PEPD comprises or consists of the sequence of SEQ ID NO:1. One unit of prolidase activity can be defined as the amount of enzyme that releases 1 μmol of proline/h under standard assay conditions. In one embodiment, an enzymatically inactive PEPD has no detectable PEPD dipeptide hydrolysis activity. In an embodiment, an enzymatically inactive PEPD comprises a G278D mutation. In embodiments, the disclosure includes any one, or any combination of PEPD mutations disclosed herein, and accordingly includes the proviso that any single or any combination of such mutants can be excluded from the invention.

PEPD used in embodiments of this disclosure can include modifications that enhance its desirable characteristics, such as the capability to bind to or enter a tumor cell or tumor microenvironment, or to enhance circulation time, bioavailability, stability, or uses related to ErbB1 or ErbB2-positive cell-targeted killing. PEPD proteins that can be used with the present disclosure include a polypeptide comprising SEQ ID NO: 1 or a modification thereof, and in embodiments also include such PEPD polypeptides within the context of a larger polypeptide.

In an embodiment, a PEPD protein can be conjugated to an immunoglobulin (Ig) or a fragment thereof to provide a chimeric PEPD/Ig molecule. Such a construct is expected to be useful in involving various aspects of the immune response of the individual to facilitate targeted killing of ErbB2+ cells. The present disclosure likewise encompasses PEPD-Fc chimeric proteins and pharmaceutical compositions comprising them. In various embodiments, the Fc region is an Fc region or fragments thereof is from an IgA, IgG, or IgE antibody, although Fc regions from other antibody types, or synthetic/artificial Fc regions can also be used. In embodiments, the Fc region is a human IgG2a or human IgG1 or a fragment of such Fc regions. The Fc region can comprise or consist of an amino acid sequence that is identical to an Fc region produced by a mammal, such as a human. In various embodiments, the Fc region may have between 80% to 100% (including all integers there between) amino acid sequence similarity to an Fc region produced by a mouse and/or a human. The Fc region may be an intact Fc region, meaning an entire Fc region, or may be a fragment of the Fc region. Those skilled in the art will recognize that the “Fc region” of an antibody means the “Fragment, crystallizable” region of the antibody, which comprises two heavy chains that contribute two or three constant domains (CD) depending on the class of the antibody. Nucleotide sequences encoding Fc regions, as well as the amino acid sequences of Fc regions for mouse and human immunoglobulins are well known in the art. In one embodiment, the Fc portion of the fusion proteins comprises only antibody heavy chain(s). Those skilled in the art will recognize that for demonstration of the invention using murine animal models, the Fc portion of the fusion protein may be an IgG2a or IgG2b Fc murine Ig portion, while for therapy and/or prophylaxis of disease in humans, the Fc portion is preferably an IgG1 or an IgG3 Fc portion. In certain embodiments, the Fc portion of the fusion proteins provided herein do not include antigen recognition portions (i.e., the antibody portion of the fusion proteins do not contain antibody variable regions). Thus, the fusion proteins are distinct from antibodies that do contain antigen binding portions. DNA constructs encoding the Fc-fusion PEPD proteins can be made using any conventional techniques well known to those skilled in the art. For example, the Fc-fusion encoding constructs can be made using commercially available reagents. For instance, INVIVOGEN offers the pFUSE-Fc family of plasmids developed to facilitate the construction of Fc-Fusion proteins by fusing a sequence encoding a given protein to the Fc region of an immunoglobulin (Ig). In this construct, the Fc region comprises the CH2 and CH3 domains of the IgG heavy chain and the hinge region. The hinge acts as a flexible spacer between the two parts of the Fc-fusion protein, which permits each part of the fusion protein to function independently if desired.

In non-limiting embodiments, the PEPD polypeptides can be combined with or coupled to a chemotherapeutic agent, or any other agent that has cytotoxic activity.

Conjugates and combinations of the PEPD protein and chemotherapeutic agents may be made using any suitable techniques.

The PEPD proteins can be provided in pharmaceutical compositions for administration by combining them with any suitable pharmaceutically acceptable carriers, excipients and/or stabilizers. Some suitable examples of pharmaceutically acceptable carriers, excipients and stabilizer can be found in Remington: The Science and Practice of Pharmacy (2005) 21 st Edition, Philadelphia, PA. Lippincott Williams & Wilkins. Further, any suitable delivery vehicle can be used in the invention, such as a controlled release delivery formulation in which the PEPD is released over a period of time. If desired, the pharmaceutical composition can comprise both PEPD and a coagulation inhibitor.

Administration of formulations comprising PEPD and the described antibody or antigen binding fragment there as described herein can be performed using any suitable route of administration, including but not limited to parenteral, intraperitoneal, intrapulmonary, oral, and intra-tumoral. Parenteral infusions include intramuscular, intravenous, intraarterial intraperitoneal, and subcutaneous administration. In an embodiment a subcutaneous administration is used.

In embodiments a single dose of a described monoclonal antibody is sufficient to enable a therapeutic effect of the PEPD. In embodiments, a single dose of Garadacimab is sufficient to inhibit degradation of PEPD for a period of at least 7 days. In embodiments, administration of Garadacimab has an improved PEPD circulating persistence time relative to a coagulation inhibitor, such as enoxaparin (EP). In embodiments, the single dose of Garadacimab is at least 10 mg. In embodiments, the single dose of Garadacimab is 10 mg-100 mgs, inclusive, and including all numbers and ranges of numbers there between. In embodiments, the single dose of Garadacimab is 10, 11, 12, 13, 13, 15, 16, 17, 18, 19, 20, 21, 22, 23, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mgs. In embodiments, the single dose of Garadacimab results in persistence of a concentration of PEPD as administered on day one of at least 50% of the PEPD for a period of 7-28 days. In embodiments, the single dose of Garadacimab results in persistence of a concentration of PEPD as administered on day of at least 50-90%, inclusive, and including all ranges and intervals of numbers there between, of the PEPD for a period of 7-28 days. In embodiments, a single dose of Garadacimab is the first dose of Garadacimab that an individual has ever had. In embodiments, a single dose of Garadacimab is a dose of Garadacimab that is administered after a period of at least one, two, three, or four weeks during which the individual received no Garadacimab. In an embodiment, Garadacimab is administered one every three weeks.

The amount PEPD and any other active agent such as Garadacimab to be included in a composition and/or to be used in the method can be determined by those skilled in the art, given the benefit of the present disclosure. In embodiments, a combination of the described PEPD an antibody is administered to an individual who has breast cancer, bladder cancer, ovarian cancer, stomach cancer, or aggressive forms of uterine cancer, such as uterine serous endometrial carcinoma, or other cancers overexpressing ErbB1 or ErbB2, or both. In embodiments, the individual who is treated with PEPD and Garadacimab does not have a disorder associated with blood clotting, a kallikrein-kinin system dysregulation, Hereditary angioedema, or a combination thereof. In embodiments the individual who is treated with PEPD and Garadacimab does not have a viral infection. In embodiments, treatment of an individual who has cancer as described herein consists of administering PEPD and Garadacimab.

The following Examples are intended to illustrate but not limit the invention.

### Example 1

This Example demonstrates that a single dose of Garadacimab blocks PEPD^{G278D} degradation in vivo.

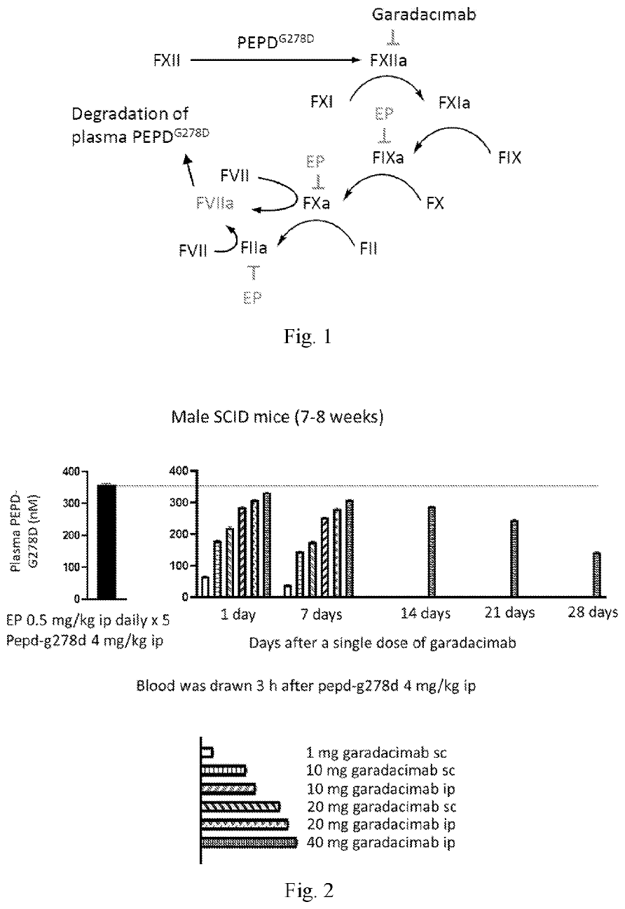

Briefly, PEPD^{G278D} upon entering blood circulation binds to and activates coagulation factor 12 (FXII). Activated FXII (FXIIa) initiates a cascade of activation of coagulation proteases, leading to activation of coagulation factor 7 (FVII), and activated FVII (FVIIa) degrades PEPD^{G278D}. It was previously discovered that enoxaparin (EP), a clinically used anticoagulant, is highly effective in blocking PEPD^{G278D} degradation by inhibiting three coagulation factors, including FIXa, FXa, and FIIa (FIG. 1). In the previous mouse studies, EP is administered either subcutaneously or intraperitoneally at 0.5 mg/kg daily, to fully block PEPD^{G278D} degradation in vivo. The EP dosing regimen increases plasma concentrations of PEPD^{G278D} by approximately 50 fold in mice. Without EP, it was not previously possible to achieve therapeutically relevant concentrations of PEPD^{G278D} in vivo. However, as shown in FIG. 1, EP does not inhibit activated FXIIa. It is known that activated FXIIa along with prekallikrein and high-molecular-weight kininogen generate bradykinin, initiates the intrinsic coagulation cascade and promotes inflammation. In addition, EP must be administered daily.

Garadacimab is an FXII/FXIIa-directed monoclonal antibody. In late 2019, US FDA granted orphan drug designation to garadacimab as an investigational therapy for prevention of bradykinin-mediated hereditary angioedema. The present disclosure unexpectedly reveals that by blocking FXIIa, garadacimab is highly effective in blocking PEPD^{G278D} degradation in vivo. Moreover, garadacimab is expected to be more suitable for use combination with PEPD^{G278D}, since it is expected to prevent bradykinin generation and may be administered less frequently, such as in a single dose to potentiate the anti-cancer effects of PEPD. Several experiments were performed to assess the ability of garadacimab to block PEPD^{G278D} degradation in vivo. Garadacimab was obtained from ProteoGenix in France and its activity was evaluated in this Example in male SCID mice (7-8 weeks of age).

Experimental design: Garadacimab was given to mice by either intraperitoneal injection (ip) or subcutaneous injection (sc) on day 1 at 1, 10, 20 or 40 mg/kg. On day 2, PEPD^{G278D} was given to mice at 4 mg/kg ip, and blood was drawn 3 h later. On day 7, another dose of PEPD^{G278D} was given to the same mice at 4 mg/kg ip, and blood was drawn 3 h later. With 40 mg/kg garadacimab ip, PEPD^{G278D} was also administered on days 14, 21, and 28, and blood was drawn 3 h later at each time point. In a previous experiment, EP was given to mice at 0.5 mg/kg ip once daily; 1 h after the fifth EP dose, PEPD^{G278D} was given to mice at 4 mg/kg ip, and blood was drawn 3 h later. Plasma concentration of PEPD^{G278D} was measured by ELISA.

### Result

With EP, plasma PEPD^{G278D} concentration at 3 h after injection is 360 nM+/−4.3 SD (n=3)

With garadacimab at 1 mg/kg sc, plasma PEPD^{G278D} concentration at 3 h after injection is 66.0 nM+/−1.2 SD (n=3) (1 day after garadacimab), and 39.1 nM+/−1.0 SD (n=3) (7 days after garadacimab).

With garadacimab at 10 mg/kg sc, plasma PEPD^{G278D} concentration at 3 h after injection is 178.7 nM+/−1.7 SD (n=3) (1 day after garadacimab), and 144.3+/−2.3 SD (n=3) (7 days after garadacimab).

With garadacimab at 10 mg/kg ip, plasma PEPD^{G278D} concentration at 3 h after injection is 220.3 nM+/−2.4 SD (n=3) (1 day after garadacimab), and 175.2+/−2.6 SD (n=3) (7 days after garadacimab).

With garadacimab at 20 mg/kg sc, plasma PEPD^{G278D} concentration at 3 h after injection is 284.4+/−2.3 SD (n=3) (1 day after garadacimab), and 252.9+/−1.6 SD (n=3) (7 days after garadacimab)

With garadacimab at 20 mg/kg ip, plasma PEPD^{G278D} concentration at 3 h after injection is 308.0 nM+/−1.9 SD (n=3) (1 day after garadacimab), and 280.0+/−1.8 SD (n=3) (7 days after garadacimab).

With garadacimab at 40 mg/kg ip, plasma PEPD^{G278D} concentration at 3 h after injection is 331.2 nM+/−2.2 SD (n=3) (1 day after garadacimab), 308.0 nM+/−1.4 SD (n=3) (7 days after garadacimab), 287.0+/−1.4 SD (14 days after garadacimab), 244.0+/−2.5 SD (21 days after garadacimab), and 141.7+/−2.3 SD (28 days after garadacimab).

Similar results are described in FIG. 2.

### Example 2

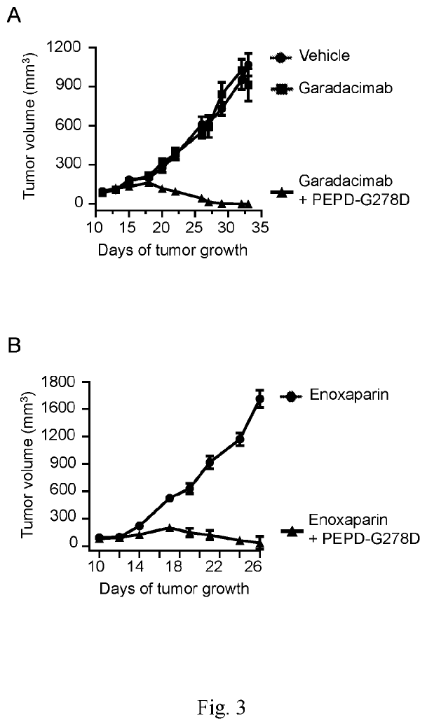

FIG. 3 demonstrates that a single dose of garadacimab is as effective as daily dose of enoxaparin in enabling the antitumor activity of PEPD-G278D in an orthotopic HER2-positive breast cancer patient-derived xenograft (PDX) model. The data was generated as described in the description of FIG. 3.

### Example 3

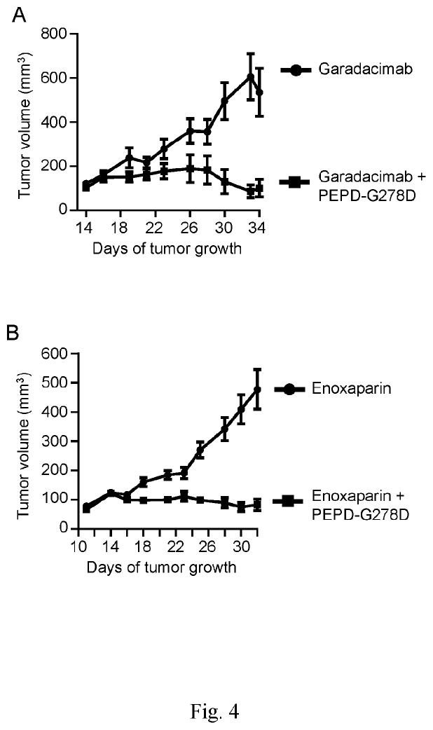

FIG. 4 demonstrates that a single dose of garadacimab is as good as daily dose of enoxaparin in enabling the antitumor activity of PEPD-G278D in orthotopic HER2-positive breast HCC1954 tumors. The data was generated as described in the description of FIG. 4.

While the invention has been described through illustrative examples, routine modifications will be apparent to those skilled in the art, which modifications are intended to be within the scope of the invention.

1. A method for therapy of cancer comprising administering to an individual in need thereof an antibody or antigen binding fragment thereof that specifically binds to Factor XII, and peptidase D (PEPD).

2. The method of claim 1, wherein the antibody that specifically binds to Factor XII is garadacimab.

3. The method of claim 2, wherein the PEPD has less dipeptide hydrolysis activity as compared to a PEPD comprising the sequence of SEQ ID NO:1.

4. The method of claim 3, wherein the PEPD comprises a mutation of glycine at position 278 of SEQ ID NO:1.

5. The method of claim 3, wherein the mutation glycine at position 278 of SEQ ID NO: 1 comprises aspartic acid at position 278 of SEQ ID NO:1.

6. The method of claim 5, wherein the cancer is ErbB1 or ErbB2 positive cancer.

7. The method of claim 6, wherein the garadacimab is administered prior to administration of the PEPD.

8. The method of claim 7, wherein the garadacimab is administered only a single time during a time period and wherein the PEPD is administered more than one time during the time period.

9. The method of claim 8, wherein the time period is at least one week.

10. The method of claim 9, wherein the time period is at least 30 days.

11. The method of claim 10, wherein the PEPD is administered at least once every 7 days.
